(19) United States
(12) Patent Application Publication
(10) Pub. No.: US 2026/0363773 A1
(43) Pub. Date: Nov. 19, 2026

(54) LASER PULSE SHAPING TO ENHANCE CONVERSION EFFICIENCY AND PROTECT FIBER OPTIC DELIVERY SYSTEM FOR DISRUPTION OF VASCULAR CALCIUM

(71) Applicant: Boston Scientific Scimed, Inc., Maple Grove, MN (US)

(72) Inventors: Daniel Massimini, Brooklyn Park, MN (US); Roger McGowan, Otsego, MN (US); Haiping Shao, Plymouth, MN (US); Christopher A. Cook, Laguna Niguel, CA (US)

(73) Assignee: Boston Scientific Scimed, Inc., Maple Grove, MN (US)

(21) Appl. No.: 19/750,356

(22) Filed: Jul. 22, 2026

Related U.S. Application Data
(63) Continuation of application No. 17/190,921, filed on Mar. 3, 2021, now Pat. No. 12,702,479.
(60) Provisional application No. 62/987,060, filed on Mar. 9, 2020.

(51) Int. Cl. A61B 18/26; A61B 17/00; A61B 18/00

(52) U.S. Cl. CPC A61B 18/26; A61B 2017/00159; A61B 2017/00185; A61B 2018/0022; A61B 2018/00369; A61B 2018/00404; A61B 2018/00702; A61B 2018/00761; A61B 2018/266

(57) ABSTRACT

A catheter system includes a power source, a controller, and a light guide. The power source generates a plurality of energy pulses. The controller controls the power source so that the plurality of energy pulses cooperate to produce a composite energy pulse having a composite pulse shape. The light guide receives the composite energy pulse. The light guide emits light energy in a direction away from the light guide to generate a plasma pulse away from the light guide. The power source can be a laser and the light guide can be an optical fiber. Each of the energy pulses has a pulse width, and the energy pulses are added to one another so that the composite energy pulse has a pulse width that is longer than the pulse width of any one of the energy pulses. At least two of the energy pulses can have the same wavelength as or a different wavelength from one another.

### CROSS-REFERENCE TO RELATED APPLICATIONS

This application is a continuation of U.S. patent application Ser. No. 17/190,921, filed Mar. 3, 2021, which claims the benefit of and priority to U.S. Provisional Application No. 62/987,060, filed on Mar. 9, 2020, the disclosures of each are incorporated in their entirety herein by reference.

### BACKGROUND

Vascular lesions within and adjacent to vessels in the body can be associated with an increased risk for major adverse events, such as myocardial infarction, embolism, deep vein thrombosis, stroke, and the like. Severe vascular lesions can be difficult to treat and achieve patency for a physician in a clinical setting.

Vascular lesions may be treated using interventions such as drug therapy, balloon angioplasty, atherectomy, stent placement, vascular graft bypass, to name a few. Such interventions may not always be ideal or may require subsequent treatment to address the lesion.

Creation of a plasma via optical breakdown of an aqueous solution typically requires a significant amount of energy in a short amount of time upon which it is converted into a therapeutic bubble and/or a therapeutic pressure wave. With sufficiently high energy and short pulse durations, there is potential to damage a distal end of a light guide used to deliver light energy to generate the plasma. A means to enhance the conversion efficiency of the light energy to (plasma) pressure wave and bubble growth would reduce the required power handling requirements of the optical delivery system. Therefore, less input energy would be required for an equivalent therapy while minimizing potential damage to the light guide.

Creation of the plasma near the distal end of a small diameter light guide as in the case of aqueous optical breakdown as one method for an intravascular lithotripsy catheter has the potential for self-damage due to its proximity to the plasma creation and/or the pressure wave, high plasma temperatures, and waterjet from collapse of the bubble, as non-exclusive examples.

### SUMMARY

The present invention is directed toward a catheter system for placement within a blood vessel having a vessel wall. The catheter system can be used for treating a treatment site within or adjacent to the vessel wall. In various embodiments, the catheter system includes a power source, a controller, and a light guide. The power source generates a plurality of energy pulses. The controller controls the power source so that the plurality of energy pulses cooperate to produce a composite energy pulse having a composite pulse shape. The light guide receives the composite energy pulse. The light guide emits light energy in a direction away from the light guide to generate a plasma pulse away from the light guide.

In some embodiments, the power source is a laser.

In certain embodiments, the light guide is an optical fiber.

In some embodiments, the catheter system further includes an inflatable balloon that encircles a distal end of the light guide.

In certain embodiments, each of the plurality of energy pulses are sub-millisecond pulses.

In some embodiments, each of the energy pulses has a pulse width, and the energy pulses are added to one another so that the composite energy pulse has a pulse width that is longer than the pulse width of any one of the energy pulses.

In certain embodiments, at least two of the plurality of energy pulses have the same wavelength as one another.

In some embodiments, at least one of the plurality of energy pulses has a wavelength that is different from the other energy pulses.

In certain embodiments, at least two of the plurality of energy pulses have pulse widths that are the same as one another.

In some embodiments, at least two of the plurality of energy pulses have pulse widths that are different from one another.

In certain embodiments, at least two of the plurality of energy pulses have light energy that is the same as one another.

In some embodiments, at least two of the plurality of energy pulses have light energy is different from one another.

In various embodiments, the plurality of energy pulses combine to generate one continuous plasma pulse away from the distal end of the light guide.

In certain embodiments, the composite energy pulse has a pulse amplitude that increases over time.

In some embodiments, the composite energy pulse has a pulse amplitude that decreases over time.

In certain embodiments, the composite energy pulse has a pulse width having a time t, the composite energy pulse having a temporal peak that occurs after time t/2.

In various embodiments, the composite energy pulse has a pulse width having a time t, the composite energy pulse having a temporal peak that occurs before time t/2.

In certain embodiments, the composite energy pulse has a pulse width having a time t, the composite energy pulse having a temporal peak that occurs approximate at time t/2.

In some embodiments, the composite energy pulse has a temporal peak that remains substantially constant over time.

In certain embodiments, the composite energy pulse generates a plurality of plasma pulses away from the distal end of the light guide. In some such embodiments, the plurality of plasma pulses are generated at different times from one another.

In some embodiments, the composite energy pulse includes two temporal peaks that are substantially similar to one another. Still further, or in the alternative, in certain embodiments, the composite energy pulse includes two temporal peaks that are different from one another.

In certain embodiments, the composite energy pulse has a pulse amplitude that generally increases over time. In other embodiments, the composite energy pulse has a pulse amplitude that generally decreases over time.

In some embodiments, the composite energy pulse has a pulse width having a time t, the composite energy pulse having a temporal peak that occurs after time t/2. Alternatively, in other embodiments, the composite energy pulse has a pulse width having a time t, the composite energy pulse having a temporal peak that occurs before time t/2. Still alternatively, in still other embodiments, the composite energy pulse has a pulse width having a time t, the composite energy pulse having a temporal peak that occurs approximately at time t/2. Further, in some such embodiments, the composite energy pulse has a temporal peak that remains substantially constant over time.

In certain embodiments, the light guide has a distal end, and the catheter system is configured to generate a pre-bubble at a distal end of the light guide. In some such embodiments, the composite energy pulse is configured to generate the pre-bubble at a distal end of the light guide. In one such embodiment, the pre-bubble is generated by electrolysis. In another such embodiment, the pre-bubble is generated by using a resistive heater. In still another such embodiment, the pre-bubble is generated with a fluid that is delivered to near the distal end of the light guide.

In some embodiments, the controller can control a timing of the composite energy pulse relative to a start of the generation of the pre-bubble. For example, in certain such embodiments, the composite energy pulse is generated greater than approximately 1 ns and less than approximately 100 ms after a start of the generation of the pre-bubble. In other such embodiments, the composite energy pulse is generated greater than approximately 100 ns and less than approximately 1 ms after a start of the generation of the pre-bubble. In still other such embodiments, the composite energy pulse is generated greater than approximately 1 us and less than approximately 10 ms after a start of the generation of the pre-bubble. In yet other such embodiments, the composite energy pulse is generated greater than approximately 5 μs and less than approximately 500 μs after a start of the generation of the pre-bubble. In still yet other such embodiments, the composite energy pulse is generated approximately 50 μs after a start of the generation of the pre-bubble.

In certain embodiments, the power source includes (i) a seed source, and (ii) an amplifier, the seed source emitting a low-power seed pulse, the amplifier being in optical communication with the seed source, the amplifier increasing the power of the seed pulse to generate an energy pulse.

In some embodiments, the power source includes (i) a plurality of seed sources, and (ii) a plurality of amplifiers, the seed sources each emitting a low-power seed pulse, the plurality of amplifiers each being in optical communication with one of the seed sources and each receiving one of the low-power seed pulses, each amplifier increasing the power of the seed pulse that is received by the respective amplifier, the plurality of amplifiers generating the plurality of energy pulses.

In certain embodiments, the power source includes (i) a plurality of seed sources, and (ii) an amplifier, the seed sources each emitting a low-power seed pulse, the amplifier being in optical communication with each of the seed sources and receiving the low-power seed pulses, the amplifier increasing the power of each of the seed pulses that is received by the amplifier, the amplifier generating the plurality of energy pulses.

In various embodiments, the catheter system further includes a hydrophobic material that is positioned near a distal end of the light guide.

In certain embodiments, the catheter system further includes a hydrophobic material that is positioned on a distal end of the light guide.

In some embodiments, the catheter system further includes a nano surface that is positioned near a distal end of the light guide.

In certain embodiments, the catheter system further includes a nano surface that is positioned on a distal end of the light guide.

In some embodiments, the nano surface is textured.

In certain applications, the present invention is also directed toward a method for treating a treatment site within or adjacent to a vessel wall, the method including the steps of: generating a plurality of energy pulses with a power source; controlling the power source with a controller so that the plurality of energy pulses cooperate to produce a composite energy pulse that is sent to a light guide, the composite energy pulse having a composite pulse shape; producing light energy that is emitted from the light guide with the composite energy pulse that is sent to the light guide; and generating a plasma pulse from the light energy away from the light guide.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic cross-sectional view of a catheter system having features of the present invention in accordance with various embodiments herein;

FIG. 2A is a simplified schematic diagram illustrating a first embodiment of a portion of the catheter system that generates a plurality of overlapping energy pulses that are sent to a light guide to generate a plasma pulse;

FIG. 2B is a simplified schematic diagram illustrating another embodiment of a portion of the catheter system that generates a plurality of non-overlapping energy pulses that are sent to the light guide to generate the plasma pulse;

FIG. 3A is a simplified schematic diagram illustrating an embodiment of a portion of the catheter system that generates a plurality of overlapping energy pulses that are sent to the light guide to generate the plurality of plasma pulses;

FIG. 3B is a simplified schematic diagram illustrating another embodiment of a portion of the catheter system that generates a plurality of non-overlapping energy pulses that are sent to the light guide to generate the plasma pulse;

FIG. 4A is a simplified graph illustrating one embodiment of a composite energy pulse having a composite pulse shape;

FIG. 4B is a simplified graph illustrating another embodiment of the composite energy pulse having another composite pulse shape;

FIG. 4C is a simplified graph illustrating yet another embodiment of the composite energy pulse having another composite pulse shape;

FIG. 5A is a simplified graph illustrating an embodiment of the composite energy pulse having another composite pulse shape;

FIG. 5B is a simplified graph illustrating another embodiment of the composite energy pulse having another composite pulse shape;

FIG. 5C is a simplified graph illustrating yet another embodiment of the composite energy pulse having another composite pulse shape;

FIG. 5D is a simplified graph illustrating still another embodiment of the composite energy pulse having another composite pulse shape;

FIG. 5E is a simplified graph illustrating another embodiment of the composite energy pulse having another composite pulse shape;

FIG. 5F is a simplified graph illustrating but another embodiment of the composite energy pulse having another composite pulse shape;

FIG. 6A is a simplified schematic diagram illustrating an embodiment of a portion of the catheter system that generates a pre-bubble;

FIG. 6B is a simplified schematic diagram illustrating another embodiment of a portion of the catheter system that generates the pre-bubble;

FIG. 6C is a simplified schematic diagram illustrating yet another embodiment of a portion of the catheter system that generates the pre-bubble; and

FIG. 6D is a simplified schematic diagram illustrating still another embodiment of a portion of the catheter system that generates the pre-bubble.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DESCRIPTION

Treatment of vascular lesions can reduce major adverse events or death in affected subjects. As referred to herein, a major adverse event is one that can occur anywhere within the body due to the presence of a vascular lesion (also sometime referred to herein as a “treatment site”). Major adverse events can include, but are not limited to, major adverse cardiac events, major adverse events in the peripheral or central vasculature, major adverse events in the brain, major adverse events in the musculature, or major adverse events in any of the internal organs.

As used herein, the treatment site can include a vascular lesion such as a calcified vascular lesion or a fibrous vascular lesion (hereinafter sometimes referred to simply as a “lesion” or “treatment site”), typically found in a blood vessel and/or a heart valve. Plasma formation can initiate a pressure wave and can initiate the rapid formation of one or more bubbles that can rapidly expand to a maximum size and then dissipate through a cavitation event that can also launch a pressure wave upon collapse. The rapid expansion of the plasma-induced bubbles can generate one or more pressure waves within a balloon fluid and thereby impart pressure waves upon the treatment site. The pressure waves can transfer mechanical energy through an incompressible balloon fluid to a treatment site to impart a fracture force on the lesion. Without wishing to be bound by any particular theory, it is believed that the rapid change in balloon fluid momentum upon a balloon wall of the inflatable balloon that is in contact with or positioned near the lesion is transferred to the lesion to induce fractures in the lesion.

Those of ordinary skill in the art will realize that the following detailed description of the present invention is illustrative only and is not intended to be in any way limiting. Other embodiments of the present invention will readily suggest themselves to such skilled persons having the benefit of this disclosure. Additionally, other methods of delivering energy to the lesion can be utilized, including, but not limited to, electric current induced plasma generation. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

As used herein, the terms “intravascular lesion”, “vascular lesion” and “treatment site” are used interchangeably unless otherwise noted. As such, the intravascular lesions and/or the vascular lesions are sometimes referred to herein simply as “lesions” and can include lesions located at or near blood vessels or heart valves.

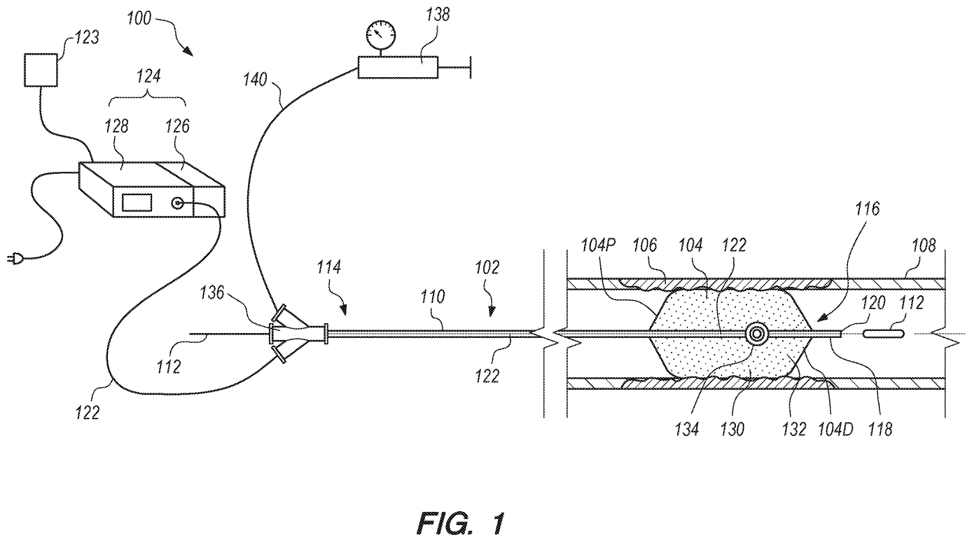

It is appreciated that the catheter systems herein can include many different forms and/or configurations other than those specifically shown and/or described herein. Referring now to FIG. 1, a schematic cross-sectional view is shown of a catheter system in accordance with various embodiments herein. A catheter system 100 is suitable for imparting pressure to induce fractures in a treatment site within or adjacent a vessel wall of a blood vessel and/or a heart valve. In the embodiment illustrated in FIG. 1, the catheter system 100 can include one or more of a catheter 102, one or more light guides 122, a controller 123, a power source 124, a manifold 136 and a fluid pump 138.

The catheter 102 includes an inflatable balloon 104 (sometimes referred to herein as “balloon”). The catheter 102 is configured to move to a treatment site 106 within or adjacent to a blood vessel 108. The treatment site 106 can include a treatment site such as a calcified vascular lesion, for example. Additionally, or in the alternative, the treatment site 106 can include a vascular lesion such as a fibrous vascular lesion.

The catheter 102 can include the balloon 104, a catheter shaft 110 and a guidewire 112. The balloon can be coupled to the catheter shaft 110. The balloon can include a balloon proximal end 104P and a balloon distal end 104D. The catheter shaft 110 can extend between a shaft proximal end 114 and a shaft distal end 116. The catheter shaft 110 can include a guidewire lumen 118 which is configured to move over the guidewire 112. The catheter shaft 110 can also include an inflation lumen (not shown). In some embodiments, the catheter 102 can have a distal end opening 120 and can accommodate and be moved over and/or along the guidewire 112 so that the balloon 104 is positioned at or near the treatment site 106.

The catheter shaft 110 of the catheter 102 can encircle one or more light guides 122 (only one light guide 122 is illustrated in FIG. 1 for clarity) in optical communication with a power source 124. The light guide 122 can be at least partially disposed along and/or within the catheter shaft 110 and at least partially within the balloon 104. In various embodiments, the light guide 122 can be an optical fiber and the power source 124 can be a laser. The power source 124 can be in optical communication with the light guide 122. In some embodiments, the catheter shaft 110 can encircle multiple light guides such as a second light guide, a third light guide, etc.

The balloon 104 can include a balloon wall 130. The balloon 104 can expand from a collapsed configuration suitable for advancing at least a portion of the catheter shaft 102 through a patient's vasculature to an expanded configuration suitable for anchoring the catheter 102 into position relative to the treatment site 106.

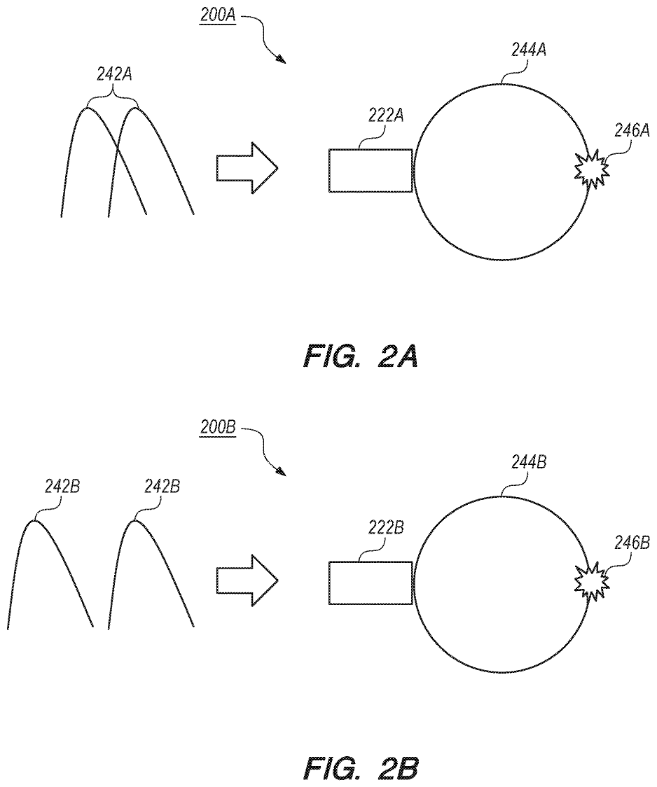

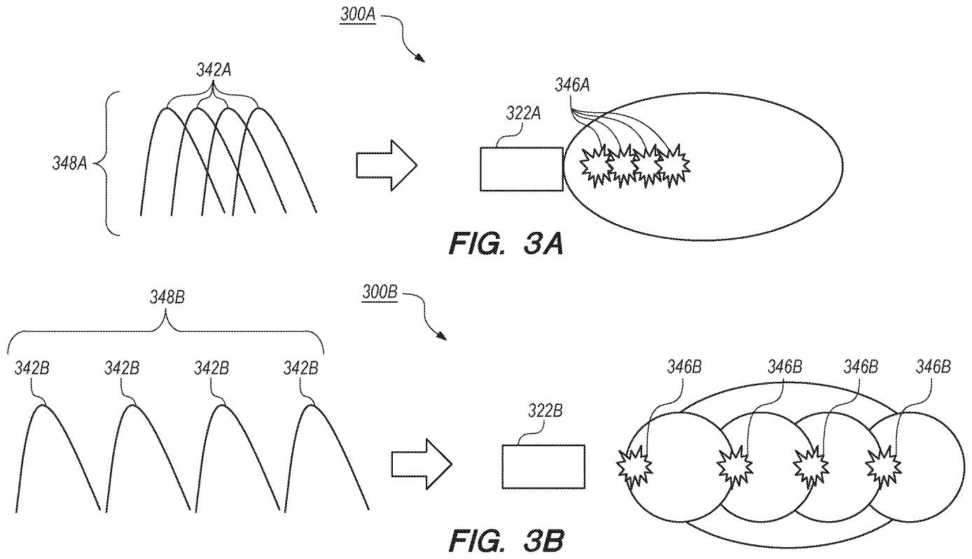

The controller 123 can control the power source 124 so that the power source can generate one or more energy pulses 242A, 242B, 342A, 342B (illustrated in FIGS. 2A-3B, for example) as provided in greater detail herein. The controller 123 may also perform other relevant functions to control operation of the catheter 102.

The power source 124 of the catheter system 100 can be configured to provide one or more sub-millisecond energy pulses that are received by the light guide 122. As provided in greater detail herein, in various embodiments, the energy pulses can combine or otherwise cooperate to produce a composite energy pulse having a composite pulse shape (not shown in FIG. 1) that is then received by the light guide 122. The light guide 122 acts as a conduit for light energy that is generated by the composite energy pulse. In certain embodiments, the power source 124 can include one or more seed sources 126 and one or more amplifiers 128. Each amplifier 128 can be in optical communication with at least one of the seed sources 126. The seed source(s) 126 can each emit a low-power seed pulse. The amplifier 128 can increase the power of the seed pulse to generate the energy pulse. In one embodiment, the power source can include one seed source 126 and one amplifier 128. Alternatively, the power source 124 can include a plurality of seed sources 126 and one amplifier 128. Still alternatively, the power source 124 can include a plurality of seed sources 126 and a plurality of amplifiers 128.

The light energy that is generated by the composite energy pulse is delivered by the light guide 122 to a location within the balloon 104. The light energy induces plasma formation in the form of a plasma pulse 134 that occurs in the balloon fluid 132 within the balloon 104. The plasma pulse 134 causes rapid bubble formation, and imparts pressure waves upon the treatment site 106. Exemplary plasma pulses 134 are shown in FIG. 1. The balloon fluid 132 can be a liquid or a gas. As provided in greater detail herein, the plasma-induced bubbles 134 are intentionally formed at some distance away from the light guide 122 so that the likelihood of damage to the light guide is decreased.

In various embodiments, the sub-millisecond pulses of light can be delivered to near the treatment site 106 at a frequency of from at least approximately 1 hertz (Hz) up to approximately 5000 Hz. In some embodiments, the sub-millisecond pulses of light can be delivered to near the treatment site 106 at a frequency from at least 30 Hz to 1000 Hz. In other embodiments, the sub-millisecond pulses of light can be delivered to near the treatment site 106 at a frequency from at least 10 Hz to 100 Hz. In yet other embodiments, the sub-millisecond pulses of light can be delivered to near the treatment site 106 at a frequency from at least 1 Hz to 30 Hz. In some embodiments, the sub-millisecond pulses of light can be delivered to near the treatment site 106 at a frequency that can be greater than or equal to 1 Hz, 2 Hz, 3 Hz, 4 Hz, 5 Hz, 6 Hz, 7 Hz, 8 Hz, or 9 Hz, 10 Hz, 20 Hz, 30 Hz, 40 Hz, 50 Hz, 60 Hz, 70 Hz, 80 Hz, 90 Hz, 100 Hz, 200 Hz, 300 Hz, 400 Hz, 500 Hz, 600 Hz, 700 Hz, 800 Hz, 900 Hz, 1000 Hz, 1250 Hz, 1500 Hz, 1750 Hz, 2000 Hz, 2250 Hz, 2500 Hz, 2750 Hz, 3000 Hz, 3250 Hz, 3500 Hz, 3750 Hz, 4000 Hz, 4250 Hz, 4500 Hz, 4750 Hz, or 5000 Hz or can be an amount falling within a range between any of the foregoing. Alternatively, the sub-millisecond pulses of light can be delivered to near the treatment site 106 at a frequency that can be greater than 5000 Hz.

It is appreciated that the catheter system 100 herein can include any number of light guides 122 in optical communication with the power source 124 at the proximal portion 114, and with the balloon fluid 132 within the balloon 104 at the distal portion 116. For example, in some embodiments, the catheter system 100 herein can include from one light guide 122 to five light guides 122. In other embodiments, the catheter system 100 herein can include from five light guides to fifteen light guides. In yet other embodiments, the catheter system 100 herein can include from ten light guides to thirty light guides. The catheter system 100 herein can include 1-30 light guides. It is appreciated that the catheter system 100 herein can include any number of light guides that can fall within a range, wherein any of the forgoing numbers can serve as the lower or upper bound of the range, provided that the lower bound of the range is a value less than the upper bound of the range. In some embodiments, the catheter system 100 herein can include greater than 30 light guides.

The manifold 136 can be positioned at or near the shaft proximal end 114. The manifold 136 can include one or more proximal end openings that can receive the one or more light guides, such as light guide 122, the guidewire 112, and/or an inflation conduit 140. The catheter system 100 can also include the fluid pump 138 that is configured to inflate the balloon 104 with the balloon fluid 132 and/or deflate the balloon 104 as needed.

As with all embodiments illustrated and described herein, various structures may be omitted from the figures for clarity and ease of understanding. Further, the figures may include certain structures that can be omitted without deviating from the intent and scope of the invention.

FIG. 2A is a simplified schematic diagram illustrating a first embodiment of a portion of the catheter system 200A that generates a plurality of overlapping energy pulses 242A. In this embodiment, the overlapping energy pulses 242A combine and are sent to a light guide 222A to generate a pre-bubble 244A and a plasma pulse 246A. The plasma pulse 246A generates pressure waves (not shown), which then disrupt the calcified lesion at or near the treatment site 106 (illustrated in FIG. 1). By combining a plurality of energy pulses 242A in a structured manner, a composite energy pulse 348A (illustrated in FIG. 3A, for example) is generated. As provided in greater detail below, in this and other embodiments, the composite energy pulse 348A can be customized or otherwise tailored to achieve a specific pre-bubble 244A and/or plasma pulse 246A.

In one embodiment, and in the embodiments which follow, the energy pulses 242A can be substantially similar in shape, amplitude and/or pulse width (duration). Alternatively, one or more of the shape, amplitude and/or duration pulse width can be different from energy pulse 242A to energy pulse 242A. With this design, the composite energy pulse can be customized in a manner that is advantageous to generating one or more plasma pulses 246A having the desired characteristics.

FIG. 2B is a simplified schematic diagram illustrating a first embodiment of a portion of the catheter system 200B that generates a plurality of separate, spaced apart energy pulses 242B. In this embodiment, the spaced apart energy pulses 242B are sent to a light guide 222B to generate a pre-bubble 244B and/or a plasma pulse 246B. The plasma pulse 246B generates pressure waves (not shown), which then disrupt the calcified lesion at or near the treatment site 106 (illustrated in FIG. 1). By using a plurality of energy pulses 242B in a structured manner, a composite energy pulse 348B (illustrated in FIG. 3B, for example) is generated. As provided in greater detail below, in this and other embodiments, the composite energy pulse 348B can be customized or otherwise tailored to achieve a specific pre-bubble 244B and/or plasma pulse 246B.

FIG. 3A is a simplified schematic diagram illustrating an embodiment of a portion of the catheter system 300A that generates a plurality of overlapping energy pulses 342A to produce a composite energy pulse 348A. The composite energy pulse 348A is sent to the light guide 322A and can generate one or more plasma pulses 346A. In this embodiment, the plasma pulses 346A can occur in relatively close proximity to one another and/or close in time to one another. In the embodiment illustrated in FIG. 3A, the plasma pulses 346A occur essentially continuously, e.g. the plasma pulses 346A are substantially in rapid-fire succession to basically create one continuous plasma pulse 346A having a longer duration than any one single plasma pulse 346A. The plasma pulses 346A can generate pressure waves (not shown), which then disrupt the calcified lesion at or near the treatment site 106 (illustrated in FIG. 1).

In one embodiment, and in the embodiments which follow, the energy pulses 342A can be substantially similar in shape, amplitude and/or pulse width (duration). Alternatively, one or more of the shape, amplitude and/or duration pulse width can be different from energy pulse 342A to energy pulse 342A.

FIG. 3B is a simplified schematic diagram illustrating an embodiment of a portion of the catheter system 300B that generates a plurality of separate, spaced apart energy pulses 342B to produce a composite energy pulse 348B. The composite energy pulse 348B is sent to the light guide 322B and can generate one or more plasma pulses 346B. In this embodiment, the plasma pulses 346B can have a greater distance between one another and/or a greater time between each plasma pulse 346B. The plasma pulses 346B can generate pressure waves (not shown), which then disrupt the calcified lesion at or near the treatment site 106 (illustrated in FIG. 1).

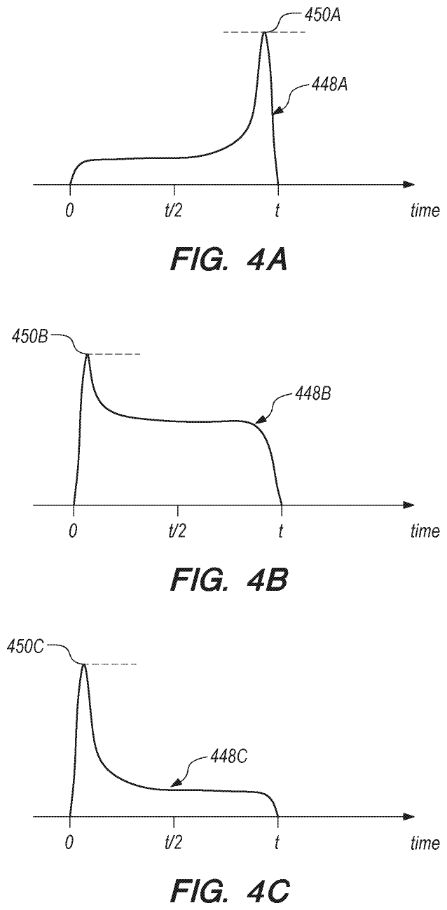

FIG. 4A is a simplified graph illustrating one embodiment of a composite energy pulse 448A having a pulse width with a duration of t. In this embodiment, the composite energy pulse 448A was formed by combining a plurality of energy pulses (illustrated in FIGS. 2A-2B and FIGS. 3A-3B, for example), as set forth in greater detail herein. In the embodiment illustrated in FIG. 4A, the composite energy pulse 448A has a temporal peak 450A (greatest amplitude) that occurs after time t/2. Further, in this embodiment, the composite energy pulse 448A has relatively low energy at the onset, which creates pre-seeding prior to the plasma pulse (not shown in FIG. 4A). In this embodiment, the composite energy pulse 448A has a greater energy toward the end of the pulse, which ultimately generates the plasma pulse.

FIG. 4B is a simplified graph illustrating one embodiment of a composite energy pulse 448B having a pulse width with a duration of t. In this embodiment, the composite energy pulse 448B was formed by combining a plurality of energy pulses (illustrated in FIGS. 2A-2B and FIGS. 3A-3B, for example), as set forth in greater detail herein. In the embodiment illustrated in FIG. 4B, the composite energy pulse 448B has a temporal peak 450B (greatest amplitude) that occurs before time t/2, resulting in the plasma pulse (not shown in FIG. 4B). Further, in this embodiment, the composite energy pulse 448B maintains a relatively high, sustaining energy after the temporal peak 450B, which can feed the plasma pulse with a relatively high energy long tail after the temporal peak 450B.

FIG. 4C is a simplified graph illustrating one embodiment of a composite energy pulse 448C having a pulse width with a duration of t. In this embodiment, the composite energy pulse 448C was formed by combining a plurality of energy pulses (illustrated in FIGS. 2A-2B and FIGS. 3A-3B, for example), as set forth in greater detail herein. In the embodiment illustrated in FIG. 4C, the composite energy pulse 448C has a temporal peak 450C (greatest amplitude) that occurs before time t/2, resulting in the plasma pulse (not shown in FIG. 4C). Further, in this embodiment, the composite energy pulse 448C maintains a relatively low, sustaining energy after the temporal peak 450C, which can feed the plasma pulse with a relatively low energy long tail after the temporal peak 450C.

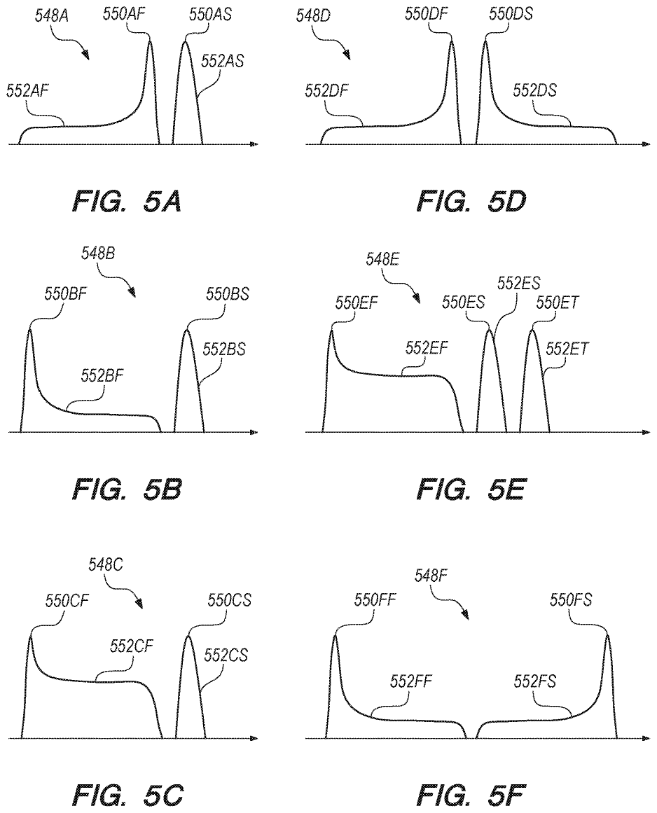

FIGS. 5A-5F illustrate non-exclusive embodiments of certain representative composite energy pulses that can be generated using the devices and methods provided herein. It is understood that these embodiments are not intended to illustrate all possible composite energy pulses, as doing so would be impossible. Rather, FIGS. 5A-5F are provided to illustrate that any composite energy pulse shape is possible using the devices and methods disclosed herein.

FIG. 5A is a simplified graph illustrating an embodiment of the composite energy pulse 548A having one composite pulse shape. In this embodiment, the composite energy pulse 548A includes two (or more) spaced apart temporal peaks such as a first temporal peak 550AF and a second temporal peak 550AS. Further, in one embodiment, the composite energy pulse 548A can have two (or more) separate, spaced apart pulses including a first pulse 552AF and a second pulse 552AS, each having a different pulse shape from one another, although it is understood that the pulse shapes can alternatively be substantially similar or identical to one another.

FIG. 5B is a simplified graph illustrating an embodiment of the composite energy pulse 548B having one composite pulse shape. In this embodiment, the composite energy pulse 548B includes two (or more) spaced apart temporal peaks such as a first temporal peak 550BF and a second temporal peak 550BS. Further, in one embodiment, the composite energy pulse 548B can have two (or more) separate, spaced apart pulses including a first pulse 552BF and a second pulse 552BS, each having a different pulse shape from one another, although it is understood that the pulse shapes can alternatively be substantially similar or identical to one another.

FIG. 5C is a simplified graph illustrating an embodiment of the composite energy pulse 548C having one composite pulse shape. In this embodiment, the composite energy pulse 548C includes two (or more) spaced apart temporal peaks such as a first temporal peak 550CF and a second temporal peak 550CS. Further, in one embodiment, the composite energy pulse 548C can have two (or more) separate, spaced apart pulses including a first pulse 552CF and a second pulse 552CS, each having a different pulse shape from one another, although it is understood that the pulse shapes can alternatively be substantially similar or identical to one another.

FIG. 5D is a simplified graph illustrating an embodiment of the composite energy pulse 548D having one composite pulse shape. In this embodiment, the composite energy pulse 548D includes two (or more) spaced apart temporal peaks such as a first temporal peak 550DF and a second temporal peak 550DS. Further, in one embodiment, the composite energy pulse 548D can have two (or more) separate, spaced apart pulses including a first pulse 552DF and a second pulse 552DS, each having a different pulse shape from one another, although it is understood that the pulse shapes can alternatively be substantially similar or identical to one another.

FIG. 5E is a simplified graph illustrating an embodiment of the composite energy pulse 548E having one composite pulse shape. In this embodiment, the composite energy pulse 548E includes three (or more) spaced apart temporal peaks such as a first temporal peak 550EF, a second temporal peak 550ES and a third temporal peak 550ET. Further, in one embodiment, the composite energy pulse 548E can have three (or more) separate, spaced apart pulses including a first pulse 552EF, a second pulse 552ES and a third pulse 552ET, so that at least two of the pulses 552EF, 552ES have different pulse shapes from one another, although it is understood that the pulse shapes can alternatively all be substantially similar or identical to one another, or still alternatively, can be all different from one another.

FIG. 5F is a simplified graph illustrating an embodiment of the composite energy pulse 548F having one composite pulse shape. In this embodiment, the composite energy pulse 548F includes two (or more) spaced apart temporal peaks such as a first temporal peak 550FF and a second temporal peak 550FS. Further, in one embodiment, the composite energy pulse 548F can have two (or more) separate, spaced apart pulses including a first pulse 552FF and a second pulse 552FS, each having a different pulse shape from one another, although it is understood that the pulse shapes can alternatively be substantially similar or identical to one another.

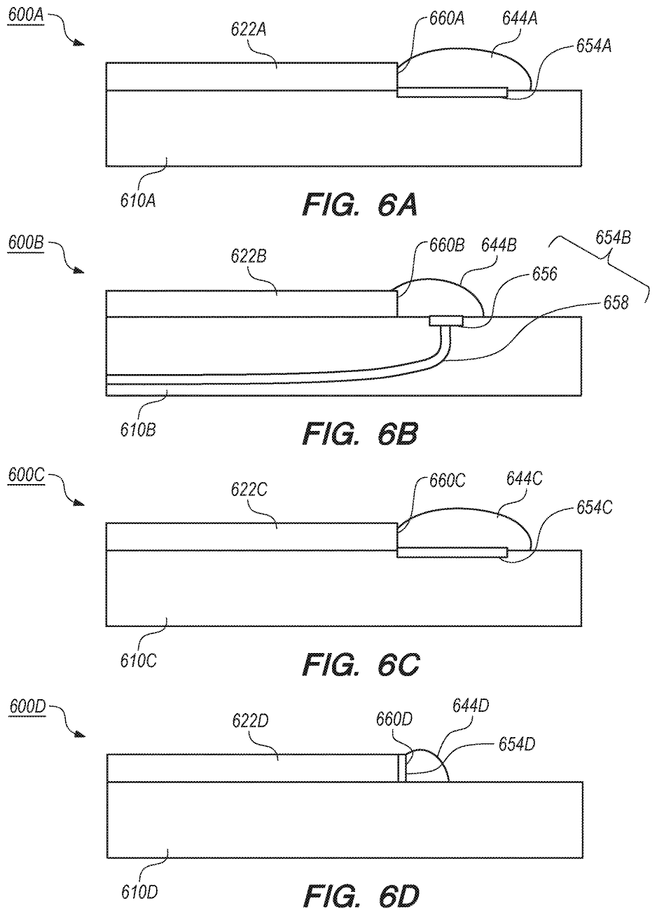

FIG. 6A is a simplified schematic diagram illustrating an embodiment of a portion of the catheter system 600A that generates a pre-bubble 644A. In this embodiment, the catheter system 600A includes a catheter shaft 610A, a light guide 622A and a pre-bubble generator 654A. The pre-bubble generator 654A generates the pre-bubble 644A to provide a gap between the light guide 622A and a plasma pulse (not shown in FIG. 6A) that will ultimately be generated. In one such embodiment, the pre-bubble generator 654A can include a resistive heater. Alternatively, or in addition, the pre-bubble generator 654A can include a pair (or more) of electrolysis electrodes or any other material that would encourage or promote generation of a pre-bubble 644A at or near a distal end 660A of the light guide 622A. With these designs, damage to the light guide 622A is inhibited because the plasma pulse does not occur immediately at or on the light guide 622A, but instead occurs away from the light guide 622A.

FIG. 6B is a simplified schematic diagram illustrating another embodiment of a portion of the catheter system 600B that generates the pre-bubble 644B. In this embodiment, the catheter system 600B includes a catheter shaft 610B, a light guide 622B and a pre-bubble generator 654B. The pre-bubble generator 654B generates the pre-bubble 644B to provide a gap between the light guide 622B and a plasma pulse (not shown in FIG. 6B) that will ultimately be generated. In one such embodiment, the pre-bubble generator 654B can include a fluid port 656 and a fluid line 658 that is in fluid communication with the fluid port 656. In this embodiment, a fluid (such as air, in one non-exclusive embodiment) can be delivered to the fluid port 656 via the fluid line 658, which can generate the pre-bubble 644B. With this design, damage to the light guide 622B is inhibited because the plasma pulse does not occur immediately at the distal end 660B or anywhere on the light guide 622B, but instead occurs away from the light guide 622B.

FIG. 6C is a simplified schematic diagram illustrating yet another embodiment of a portion of the catheter system 600C that generates the pre-bubble 644C. In this embodiment, the catheter system 600C includes a catheter shaft 610C, a light guide 622C and a pre-bubble generator 654C. The pre-bubble generator 654C generates the pre-bubble 644C to provide a gap between the light guide 622C and a plasma pulse (not shown in FIG. 6C) that will ultimately be generated. In one such embodiment, the pre-bubble generator 654C can include a hydrophobic coating. In this embodiment, surface tension is created so that the pre-bubble would self-form due to hydrophobicity forces. Alternatively, or in addition, the pre-bubble generator 654C can include a nano-textured surface or any other surface or material that would encourage or promote generation of a pre-bubble at or near a distal end 660C of the light guide 622C. In this embodiment, the pre-bubble generator 654C is positioned on the catheter shaft 610C. However, it is recognized that the pre-bubble generator 654C can be positioned at or on another structure within the catheter system 600C. With this design, damage to the light guide 622C is inhibited because the plasma pulse does not occur immediately at or on the light guide 622C, but instead occurs away from the light guide 622C.

FIG. 6D is a simplified schematic diagram illustrating still another embodiment of a portion of the catheter system 600D that generates the pre-bubble 644D. In this embodiment, the catheter system 600D includes a catheter shaft 610D, a light guide 622D and a pre-bubble generator 654D. The pre-bubble generator 654D generates the pre-bubble 644D to provide a gap between the light guide 622D and a plasma pulse (not shown in FIG. 6D) that will ultimately be generated. In one such embodiment, the pre-bubble generator 654D can include a hydrophobic coating. Alternatively, or in addition, the pre-bubble generator 654D can include a nano-textured surface or any other surface or material that would encourage or promote generation of a pre-bubble at or near a distal end 660D of the light guide 622D. In this embodiment, the pre-bubble generator 654D is positioned on the light guide 622D. However, it is recognized that the pre-bubble generator 654D can be positioned at or on another structure within the catheter system 600D. With this design, damage to the light guide 622D is inhibited because the plasma pulse does not occur immediately at or on the light guide 622D, but instead occurs away from the light guide 622D.

### Light Guides

The light guides illustrated and/or described herein can include an optical fiber or flexible light pipe. The light guides illustrated and/or described herein can be thin and flexible and can allow light signals to be sent with very little loss of strength. The light guides illustrated and/or described herein can include a core surrounded by a cladding about its circumference. In some embodiments, the core can be a cylindrical core or a partially cylindrical core. The core and cladding of the light guides can be formed from one or more materials, including but not limited to one or more types of glass, silica, or one or more polymers. The light guides may also include a protective coating, such as a polymer. It is appreciated that the index of refraction of the core will be greater than the index of refraction of the cladding.

Each light guide can guide light along its length to a distal portion having at least one optical window. The light guides can create a light path as portion of an optical network including a power source. The light path within the optical network allows light to travel from one part of the network to another. Either or both of the optical fiber or the flexible light pipe can provide a light path within the optical networks herein.

The light guides illustrated and/or described herein can assume many configurations about the catheter shaft of the catheters illustrated and/or described herein. In some embodiments, the light guides can run parallel to the longitudinal axis of the catheter shaft of the catheter. In some embodiments, the light guides can be disposed spirally or helically about the longitudinal axis of the catheter shaft of the catheter. In some embodiments, the light guides can be physically coupled to the catheter shaft. In other embodiments, the light guides can be disposed along the length of the outer diameter of the catheter shaft. In yet other embodiments the light guides herein can be disposed within one or more light guide lumens within the catheter shaft. Various configurations for the catheter shafts and light guide lumens will be discussed below.

### Power Sources

The power sources suitable for use herein can include various types of power sources including lasers and lamps. Suitable lasers can include short pulse lasers on the sub-millisecond timescale. In some embodiments, the power source can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths and energy levels that can be employed to achieve plasma in the balloon fluid of the catheters illustrated and/or described herein. In various embodiments, the pulse widths can include those falling within a range including from at least 10 ns to 200 ns. In some embodiments, the pulse widths can include those falling within a range including from at least 20 ns to 100 ns. In other embodiments, the pulse widths can include those falling within a range including from at least 1 ns to 5000 ns.

Exemplary nanosecond lasers can include those within the UV to IR spectrum, spanning wavelengths of about 10 nanometers to 1 millimeter. In some embodiments, the power sources suitable for use in the catheter systems herein can include those capable of producing light at wavelengths of from at least 750 nm to 2000 nm. In some embodiments, the power sources can include those capable of producing light at wavelengths of from at least 700 nm to 3000 nm. In some embodiments, the power sources can include those capable of producing light at wavelengths of from at least 100 nm to 10 micrometers (μm). Nanosecond lasers can include those having repetition rates of up to 200 kHz. In some embodiments, the laser can include a Q-switched thulium: yttrium-aluminum-garnet (Tm:YAG) laser. In some embodiments, the laser can include a neodymium: yttrium-aluminum-garnet (Nd:YAG), holmium: yttrium-aluminum-garnet (Ho:YAG), erbium: yttrium-aluminum-garnet (Er:YAG), excimer laser, helium-neon laser, carbon dioxide laser, as well as doped, pulsed, fiber lasers.

### Pressure Waves

The catheters illustrated and/or described herein can generate pressure waves having maximum pressures in the range of at least 1 megapascal (MPa) to 100 MPa. The maximum pressure generated by a particular catheter will depend on the power source, the absorbing material, the bubble expansion, the propagation medium, the balloon material, and other factors. In some embodiments, the catheters illustrated and/or described herein can generate pressure waves having maximum pressures in the range of at least 2 MPa to 50 MPa. In other embodiments, the catheters illustrated and/or described herein can generate pressure waves having maximum pressures in the range of at least 2 MPa to 30 MPa. In yet other embodiments, the catheters illustrated and/or described herein can generate pressure waves having maximum pressures in the range of at least 15 MPa to 25 MPa. In some embodiments, the catheters illustrated and/or described herein can generate pressure waves having peak pressures of greater than or equal to 1 MPa, 2 MPa, 3 MPa, 4 MPa, 5 MPa, 6 MPa, 7 MPa, 8 MPa, 9 MPa, 10 MPa, 11 MPa, 12 MPa, 13 MPa, 14 MPa, 15 MPa, 16 MPa, 17 MPa, 18 MPa, 19 MPa, 20 MPa, 21 MPa, 22 MPa, 23 MPa, 24 MPa, 25 MPa, 26 MPa, 27 MPa, 28 MPa, 29 MPa, 30 MPa, 31 MPa, 32 MPa, 33 MPa, 34 MPa, 35 MPa, 36 MPa, 37 MPa, 38 MPa, 39 MPa, 40 MPa, 41 MPa, 42 MPa, 43 MPa, 44 MPa, 45 MPa, 46 MPa, 47 MPa, 48 MPa, 49 MPa, or 50 MPa,. It is appreciated that the catheters illustrated and/or described herein can generate pressure waves having operating pressures or maximum pressures that can fall within a range, wherein any of the forgoing numbers can serve as the lower or upper bound of the range, provided that the lower bound of the range is a value less than the upper bound of the range.

Therapeutic treatment can act via a fatigue mechanism or a brute force mechanism. For a fatigue mechanism, operating pressures would be about at least 0.5 MPa to 2 MPa, or about 1 MPa. For a brute force mechanism, operating pressures would be about at least 20 MPa to 30 MPa, or about 25 MPa. Pressures between the extreme ends of these two ranges may act upon a treatment site using a combination of a fatigue mechanism and a brute force mechanism.

The pressure waves described herein can be imparted upon the treatment site from a distance within a range from at least 0.1 millimeters (mm) to 25 mm extending radially from a longitudinal axis of a catheter placed at a treatment site. In some embodiments, the pressure waves can be imparted upon the treatment site from a distance within a range from at least 10 mm to 20 mm extending radially from a longitudinal axis of a catheter placed at a treatment site. In other embodiments, the pressure waves can be imparted upon the treatment site from a distance within a range from at least 1 mm to 10 mm extending radially from a longitudinal axis of a catheter placed at a treatment site. In yet other embodiments, the pressure waves can be imparted upon the treatment site from a distance within a range from at least 1.5 mm to 4 mm extending radially from a longitudinal axis of a catheter placed at a treatment site. In some embodiments, the pressure waves can be imparted upon the treatment site from a range of at least 2 MPa to 30 MPa at a distance from 0.1 mm to 10 mm. In some embodiments, the pressure waves can be imparted upon the treatment site from a range of at least 2 MPa to 25 MPa at a distance from 0.1 mm to 10 mm. In some embodiments, the pressure waves can be imparted upon the treatment site from a distance that can be greater than or equal to 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, or 10 mm, or can be an amount falling within a range between any of the foregoing.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

As used herein, the recitation of numerical ranges by endpoints shall include all numbers subsumed within that range, inclusive (e.g., 2 to 8 includes 2, 2.1, 2.8, 5.3, 7, 8, etc.).

It is recognized that the figures shown and described are not necessarily drawn to scale, and that they are provided for ease of reference and understanding, and for relative positioning of the structures.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions and sub-combinations thereof. It I is therefore intended that the following appended claims and claims hereafter introduced are interpreted to include all such modifications, permutations, additions and sub-combinations as are within their true spirit and scope, and no limitations are intended to the details of construction or design herein shown.

1. (canceled)

2. An intravascular catheter system comprising:
a power source configured to generate a plurality of discrete energy pulses;
a controller configured to control generation of the plurality of discrete energy pulses so that the plurality of discrete energy pulses are temporally spaced apart from one another and together define a composite energy pulse having a composite pulse shape; and
an energy guide configured to emit energy away from the energy guide and corresponding to the composite energy pulse,
wherein the energy emitted from the energy guide generates a pressure wave at one or both of a desired time and a desired location.

3. The intravascular catheter system of claim 2, further comprising an expandable member containing a fluid and wherein a distal portion of the energy guide is positioned within the expandable member.

4. The intravascular catheter system of claim 3, wherein the energy emitted from the energy guide generates a plasma in the fluid, and wherein the plasma generates the pressure wave.

5. The intravascular catheter system of claim 2, wherein the plurality of discrete energy pulses includes a first energy pulse having a first energy peak including a first amplitude and a second energy pulse having a second energy peak including a second amplitude.

6. The intravascular catheter system of claim 5, wherein the first amplitude is substantially similar to the second amplitude.

7. The intravascular catheter system of claim 5, wherein the first amplitude is different than the second amplitude.

8. The intravascular catheter system of claim 5, wherein the controller is configured to control a temporal spacing between the firing of the first energy pulse and the firing of the second energy pulse.

9. The intravascular catheter system of claim 5, wherein the first energy pulse includes a first pulse shape and the second energy pulse includes a second pulse shape different than the first pulse shape.

10. The intravascular catheter system of claim 2, wherein the plurality of discrete energy pulses generate a plurality of plasma pulses away from a distal end of the energy guide at different times from one another.

11. A console for an intravascular catheter system, the console comprising:
a power source configured to generate a plurality of discrete energy pulses; and
a controller configured to control generation of the plurality of discrete energy pulses so that the plurality of discrete energy pulses are temporally spaced apart from one another and together define a composite energy pulse having a composite pulse shape,
wherein the composite pulse shape is configured to cause the composite energy pulse to generate a pressure wave at one or both of a desired time and a desired location.

12. The console of claim 11, wherein the controller is configured to control a temporal spacing between a first energy pulse of the plurality of discrete energy pulses and a second energy pulse of the plurality of discrete energy pulses, and wherein controlling the temporal spacing defines the composite pulse shape.

13. The console of claim 11, wherein the plurality of discrete energy pulses includes a first energy pulse having a first energy peak including a first amplitude and a second energy pulse having a second energy peak including a second amplitude.

14. The intravascular catheter system of claim 13, wherein the first amplitude is substantially similar to the second amplitude.

15. The intravascular catheter system of claim 13, wherein the first amplitude is different than the second amplitude.

16. An intravascular catheter system comprising:
a power source configured to generate a plurality of discrete energy pulses;
a controller configured to control generation of the plurality of discrete energy pulses; and
an energy guide configured to emit energy away from the energy guide and corresponding to the plurality of discrete energy pulses,
wherein the plurality of discrete energy pulses are configured to generate a pre-bubble at a distal end of the energy guide and then a plasma pulse spaced from the distal end of the energy guide.

17. The intravascular catheter system of claim 16, further comprising an expandable member containing a fluid and wherein a distal portion of the energy guide is positioned within the expandable member.

18. The intravascular catheter system of claim 16, wherein a first energy pulse of the plurality of discrete energy pulses generates the pre-bubble, and a second energy pulse of the plurality of discrete energy pulses, subsequent to the first energy pulse, generates the plasma pulse, wherein the first energy pulse has a first pulse shape different from a second pulse shape of the second energy pulse.

19. The intravascular catheter system of claim 16, wherein the controller is configured to control a timing of the generation of the plasma pulse relative to a start of the generation of the pre-bubble.

20. The intravascular catheter system of claim 19, wherein the pre-bubble is generated before the plasma pulse.

21. The intravascular catheter system of claim 16, wherein the pre-bubble defines a gap between the distal end of the energy guide and the plasma pulse.
